(19) United States
(12) Patent Application Publication
(10) Pub. No.: US 2025/0057897 A1
(43) Pub. Date: Feb. 20, 2025

(54) Methods for the Treatment of Constipation

(71) Applicant: SERVATUS LTD, Queensland (AU)

(72) Inventor: Wayne Finlayson, Queensland (AU)

(21) Appl. No.: 18/715,169

(22) PCT Filed: Dec. 2, 2022
(86) PCT No.: PCT/AU2022/051445

§ 371 Date: May 31, 2024

(30) Foreign Application Priority Data
Dec. 2, 2021 (AU) 2021903906
Aug. 29, 2022 (AU) 2022902480

(51) Int. Cl. A61K 35/747; A61P 1/10

(52) U.S. Cl. CPC A61K 35/747; A61P 1/10

(57) ABSTRACT

Provided herein are methods for treating constipation, or at least one symptom of constipation, optionally idiopathic constipation, comprising administering to a subject an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

### FIELD OF THE ART

The present disclosure relates generally to methods to aid in the treatment, and alleviation of symptoms, of constipation, comprising the administration of compositions comprising one or more microorganisms, or culture supernatants or cell free filtrates derived from culture media in which the one or more microorganisms have been cultured.

### BACKGROUND

Constipation is a disorder of the gastrointestinal tract that results in infrequent passing of stool and/or the passing of hard stools leading to painful and difficult bowel motions. Constipation may be referred to as either primary or secondary constipation. Secondary constipation typically occurs in response to the use of medications such as opioids, or may be a complication resulting from another condition such as hypothyroidism. Primary constipation is classified as either idiopathic constipation or constipation-predominant irritable bowel syndrome.

Functional constipation poses a significant health and economic burden across the community with numerous studies reporting up to nearly a quarter of the adult population in Australia affected by constipation (Werth et al., 2019, BMC Gastroenterol 19:75). The burden of constipation within the community varies widely based a multitude of factors including gender, age, socioeconomic status, culture, diet and environment (Peppas et al., 2008, BMC Gastroenterol 8:5). There is a low rate of individuals reporting constipation to their healthcare practitioner, with many attempting to self-manage their symptoms through supplementation and/or over the counter laxative use. A recent study in Australia reported that of 2024 participants, 37% had indicated symptoms of constipation and the need to use laxatives to achieve a bowel motion, however, only 13% from this group consulted their healthcare practitioner for advice (Werth et al., 2019, ibid). Functional constipation is associated with a significant burden on the patient, impacting their quality of life both physically and mentally, and also economically due to the need for supplements and treatment.

Idiopathic constipation and constipation-predominant irritable bowel syndrome each fall into the category of functional constipation, a chronic condition without a readily identifiable etiology. Estimates suggest that functional constipation affects up to a quarter of the adult population, placing a significant physical and mental burden on sufferers. Functional constipation can be further classified as normal-transit constipation, slow-transit constipation or as a defecation disorder. Normal transit constipation is considered to be the most common form of functional constipation, in which stool transverses the gut at a normal rate but patients report symptoms such as hard stool or difficulty with defecating, often associated with bloating and abdominal discomfort or pain.

Many sufferers of constipation do not consult a healthcare practitioner, instead attempting to self-manage the condition with the use of over the counter supplements and medications. Most commonly, laxatives are administered. Laxative use may be indicated initially depending on the severity of symptoms, although use should temporary, typically for less than a week. Long term use of laxatives can lead to electrolyte imbalance and digestive problems including an exacerbation of constipation, and is not recommended. Laxative use is also associated with side effects such as nausea, distension, abdominal pain and vomiting.

Lifestyle adjustments including improved diet and increased consumption of dietary fibre may also be prescribed in the treatment of chronic constipation. Consumption of dietary fibre in the form of insoluble fibres, such as wheat bran or psyllium husk, is considered to have a laxative effect through bulking action, thereby promoting increased gastrointestinal transit. However, the treatment of patients with functional constipation with dietary fibre may worsen symptoms of bloating and abdominal pain, particularly in patients with constipation-predominant irritable bowel syndrome where rapid fermentation of some fibres (i.e. short chain carbohydrates) can result in excess production of gas.

While numerous options exist for the treatment of functional constipation, a large proportion of sufferers are dissatisfied with their treatment due to either safety concerns or a lack of efficacy. There remains a need for the development of new treatments for constipation.

### SUMMARY OF THE DISCLOSURE

One aspect of the present disclosure provides a method for treating constipation in a subject, comprising administering to the subject an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

The constipation may be functional constipation. The constipation may be chronic constipation.

The subject may have idiopathic constipation or constipation-predominant irritable bowel syndrome.

In a particular embodiment, the method comprises administering a composition comprising Lactobacillus paracasei, Lactobacillus zeae and Lactobacillus diolivorans.

The method may comprise administering a composition comprising Lactobacillus paracasei, Lactobacillus zeae and Lactobacillus diolivorans in the form of a liquid or solid unit dosage form, a food or a beverage.

The method may further comprise administering one or more additional agents, such as one or more Lactobacillus species optionally selected from Lactobacillus buchneri, Lactobacillus rapi, Lactobacillus plantarum, and Lactobacillus parafarraginis, or a culture supernatant or cell free filtrate derived from culture media in which said Lactobacillus species has been cultured.

Another aspect of the present disclosure provides a method for treating at least one symptom of constipation in a subject with constipation, comprising administering to the subject an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

The at least one symptom of constipation may comprise abdominal discomfort, abdominal pain, abdominal bloating, abdominal cramping, painful bowel movements, rectal burning during or after a bowel movement, incomplete bowel movements, straining to pass a bowel movement and/or inability to pass a bowel movement.

The constipation may be functional constipation. The constipation may be chronic constipation.

The subject may have idiopathic constipation or constipation-predominant irritable bowel syndrome.

In a particular embodiment, the method comprises administering a composition comprising Lactobacillus paracasei, Lactobacillus zeae and Lactobacillus diolivorans.

The method may comprise administering a composition comprising Lactobacillus paracasei, Lactobacillus zeae and Lactobacillus diolivorans in the form of a liquid or solid unit dosage form, a food or a beverage.

The method may further comprise administering one or more additional agents, such as one or more Lactobacillus species optionally selected from Lactobacillus buchneri, Lactobacillus rapi, Lactobacillus plantarum, and Lactobacillus parafarraginis, or a culture supernatant or cell free filtrate derived from culture media in which said Lactobacillus species has been cultured.

Another aspect of the present disclosure provides a composition comprising Lactobacillus paracasei, Lactobacillus zeae and Lactobacillus diolivorans, or culture supernatant(s) or cell free filtrate(s) derived from culture media in which said Lactobacillus species have been cultured, when used for treating constipation or at least one symptom thereof.

In accordance with aspects and embodiments of the present disclosure, the method may comprise administering to the subject a microbial biotherapeutic composition comprising Lactobacillus paracasei, Lactobacillus zeae and Lactobacillus diolivorans. The microbial biotherapeutic composition may be administered in the form of, for example, a liquid or solid unit dosage form, a food or a beverage.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “effective amount” includes within its meaning a non-toxic but sufficient amount of a composition to provide the desired effect and/or physiological response. The exact amount required will vary from subject to subject depending on factors such as the species being treated, the age and general condition of the subject, the severity of the condition being treated, the particular agent being administered and the mode of administration and so forth. For any given case, an appropriate “effective amount” may be determined by one of ordinary skill in the art using only routine experimentation.

As used herein the terms “treating”, “treatment” and the like refer to any and all applications which remedy, or otherwise hinder, retard, or reverse the progression of, constipation, or at least one symptom of constipation, including reducing the severity of constipation. Treatment in accordance with the present disclosure may result in resolution of the constipation or of symptoms associated with the constipation. Alternatively, treatment may result in an improvement in one of more symptoms of constipation. Thus, treatment does not necessarily imply that a subject is treated until complete resolution of, or recovery from, constipation.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

In the context of this specification, the term “microbial biotherapeutic” is to be given its broadest construction and is understood to refer to a microbial cell population or preparation, or component of a microbial cell population or preparation, which when administered to a subject in an effective amount promotes a health benefit in the subject. The cell population may comprise bacteria from one species or strain, or alternatively from multiple species or strains.

In the context of this specification, the term “prebiotic” is to be given its broadest construction and is understood to refer to any non-digestible substance that stimulates the growth and/or activity of commensal beneficial bacteria in the digestive system.

In the context of this specification, the terms “food” and “beverage” include but are not limited to health foods and beverages, functional foods and beverages, and foods and beverages for specified health use. When such foods or beverages of the present invention are used for subjects other than humans, the terms can be used to include a feedstuff.

Provided herein are methods for treating constipation wherein a subject is administered an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

Also provided herein are methods for treating at least one symptom of constipation wherein a subject with constipation is administered an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

Subjects in need of treatment in accordance with the present disclosure may be diagnosed as having constipation by any method known in the art. Diagnosis may involve clinical assessment by a medical practitioner (including, for example, physical examination, or X-ray) and/or the completion of one or more completion of self-reporting questionnaires. For example, a diagnosis of constipation may be made using the Rome III Diagnostic Criteria (Longstreth et al., 2006, Gastroenterol 130:1480-1491) or the Rome IV Diagnostic Criteria (Lacy et al., 2016, Gastroenterol 150:1393-1407). The skilled addressee will recognise that the scope of the present disclosure is not limited by reference to any specific diagnostic measure of constipation.

A subject to be treated in accordance with the present disclosure may be suffering from acute or chronic constipation. The constipation may be functional constipation, spastic constipation, atonic constipation, constipation accompanying irritable bowel syndrome, organic constipation, constipation accompanying enteroparalytic ileus, constipation accompanying congenital digestive tract dysfunction and constipation accompanying ileus, or combinations thereof. In particular embodiments the constipation may be functional constipation. In particular embodiments the subject may have idiopathic constipation or constipation-predominant irritable bowel syndrome.

Methods of the present disclosure may treat or improve one or more symptoms of constipation in a subject with constipation. Such symptoms may include, for example, abdominal discomfort, abdominal pain, abdominal bloating, abdominal cramping, painful bowel movements, rectal burning during or after a bowel movement, incomplete bowel movements, straining to pass a bowel movement and/or inability to pass a bowel movement. Symptoms may also include those affecting the day to day quality of life of a subject with constipation, including for example the feeling of physical, emotional and/or psychosocial discomfort associated with bowel movements, and irritability, embarrassment and/or stress associated with quality or frequency of bowel movements.

In accordance with the present disclosure, treatment of constipation may comprise an improvement or resolution of constipation, or improvement or resolution of at least one symptom of constipation. The resolution or improvement of constipation or symptoms of constipation may be determined by any means known to those skilled in the art. This may include, for example, clinical assessment by a medical practitioner (including, for example, physical examination, or X-ray) and/or the completion of one or more self-reporting questionnaires. For example, subjects may complete the Patient Assessment of Constipation-Symptoms (PAC-SYM) questionnaire (Frank et al., 1999, Scand J Gastroenterol 34:870-877) and/or the Patient Assessment of Constipation-Quality of Life (PAC-QOL) questionnaire (Dubois et al., 2010, Neurogastroenterol Motil 22 e54-63). PAC-SYM assesses the severity of patient-reported symptoms, and is divided into three symptom groups, abdominal, rectal and stool, each scored on a 5-point scale of severity. The PAC-QOL provides a measure of the impact and burden of constipation on a constipated patient's everyday life. Questions are categorised to 4 items on physical discomfort, 8 items on psychosocial discomfort, 5 items on treatment satisfaction, and 11 items on worries and discomfort, each scored on a 5-point scale of severity. For each of PAC-SYM and PAC-QOL, a determination of positive response can be obtained by determination of the minimal important difference (MID) (see, e.g. Yiannakou et al., 2017, Ailment Pharmacol Ther 46:1103-1111; Marquis et al., 2005, Scand J Gastroenterol 40:540-551). The skilled person will appreciate that the scope of the present disclosure is not limited by reference to any specific means of assessing or determining improvements in constipation, bowel movements or constipation symptoms in a subject.

In an embodiment, treatment of constipation in accordance with the present disclosure may comprise an improvement in one or more of the parameters assessed in PAC-SYM. In another embodiment, In an embodiment, treatment of constipation in accordance with the present disclosure may comprise an improvement in one or more of the parameters assessed in PAC-QOL.

In some embodiments the methods of the present disclosure comprise administering an effective amount of a composition as described herein in one or more doses per day, optionally one or two doses per day. For example the composition may be administered in a single dose in the morning or evening, or in two doses spaced apart through the day.

Typically administration of compositions in accordance with methods of the present disclosure is continued at least until at least one symptom of constipation has improved. Administration may be continued, for example, for about 7 days, 10 days, 14 days, 21 days, four weeks, five weeks, six weeks, seven weeks, two months, three months, four months, five months, six months or longer. The dosing regimen may be modified or altered during the course of administration as needed by the subject or on the advice of a medical practitioner or other health care professional.

The methods of the present disclosure contemplate the administration of Lactobacillus paracasei, Lactobacillus zeae and Lactobacillus diolivorans or culture supernatant(s) or cell free filtrate(s) derived from culture media in which said Lactobacillus species have been cultured. In view of some taxonomic discrepancies and uncertainties, L. zeae may also be referred to elsewhere as L. casei. However this is not settled, and L. zeae can be regarded as distinct (see http://lactotax.embl.de/wuyts/lactotax/). For the purposes of the present disclosure the L. zeae nomenclature is retained.

In particular embodiments the methods of the present disclosure contemplate the administration of the Lactobacillus paracasei, Lactobacillus zeae and Lactobacillus diolivorans in the same composition.

The Lactobacillus paracasei may be L. paracasei SVT-09 (which may be elsewhere referred to by the alternate designation SVT 04P1) deposited pursuant to the Budapest Treaty with the Belgian Coordinated Collections of Microorganisms (BCCM) on 27 Feb. 2019 under Accession Number LMG P-31290.

The Lactobacillus zeae may be L. zeae SVT 08Z1, deposited pursuant to the Budapest Treaty with the Belgian Coordinated Collections of Microorganisms (BCCM) on 27 Feb. 2019 under Accession Number LMG P-31295, and previously described in WO 2020/073088.

The Lactobacillus diolivorans may be L. diolivorans SVT-03 (which may be elsewhere referred to by the alternate designation SVT 01D1) deposited pursuant to the Budapest Treaty with the Belgian Coordinated Collections of Microorganisms (BCCM) on 27 Feb. 2019 under Accession Number LMG P-31287.

In the following discussion, in the context of administration of the Lactobacillus species or culture supernatants or cell free filtrates derived from culture media in which the Lactobacillus has been cultured, and in the context of compositions comprising the same, the term “Lactobacillus” may be used hereinafter to refer to bacterial cells (typically live cells) of the specific Lactobacillus species defined herein per se, as well as to culture supernatants or cell free filtrates derived from culture media in which the specific Lactobacillus species defined herein have been cultured. In embodiments in which culture supernatants or cell free filtrates derived from culture media in which said Lactobacillus have been cultured are employed, the bacteria of each species may be cultured together or individually, or some may be cultured together and others may be cultured individually. Hence multiple culture supernatants or cell free filtrates may be combined.

Also contemplated herein are combinations wherein bacterial cells of one or more of the Lactobacillus species defined herein are combined with culture supernatant(s) or cell free filtrate(s) derived from culture media in which one or more of the Lactobacillus species defined herein have been cultured.

Methods of the present disclosure may further comprise the administration of one or more of Lactobacillus buchneri, Lactobacillus rapi, Lactobacillus plantarum, and Lactobacillus parafarraginis, or culture supernatants or cell free filtrates derived from culture media in which said Lactobacillus have been cultured. In such embodiments the bacteria may be cultured together or separately.

The Lactobacillus parafarraginis may be L. parafarraginis SVT-18 (which may be elsewhere referred to by the alternate designation SVT 05P2) deposited pursuant to the Budapest Treaty with the Belgian Coordinated Collections of Microorganisms (BCCM) on 27 Feb. 2019 under Accession Number LMG P-31292.

The Lactobacillus buchneri may be L. buchneri SVT-23 (which may be elsewhere referred to by the alternate designation SVT 06B1) deposited pursuant to the Budapest Treaty with the Belgian Coordinated Collections of Microorganisms (BCCM) on 27 Feb. 2019 under Accession Number LMG P-31293.

The Lactobacillus rapi may be L. rapi SVT-24 (which may be elsewhere referred to by the alternate designation SVT 07R1) deposited pursuant to the Budapest Treaty with the Belgian Coordinated Collections of Microorganisms (BCCM) on 27 Feb. 2019 under Accession Number LMG P-31294.

The Lactobacillus plantarum may be L. plantarum SVT 09P1, deposited pursuant to the Budapest Treaty with the Belgian Coordinated Collections of Microorganisms (BCCM), Federal Public Planning Service Science Policy, 8 rue de la Science B-1000, Brussels, Belgium, on 10 Aug. 2020 under Accession Number LMG P-31923.

The concentrations of individual Lactobacillus species to be administered in accordance with methods of the present disclosure will depend on a variety of factors including the identity and number of individual species employed, the general health and wellbeing of the subject, the severity of constipation to be treated and the form and route in which a composition is administered For any given case, appropriate concentrations may be determined by one of ordinary skill in the art using only routine experimentation. By way of example only, the concentration of the Lactobacillus species, or each species present in the case of a combination, may be from about 1×10² cfu/ml to about 1×10¹¹ cfu/ml, and may be about 1×10³ cfu/ml, about 2.5×10³ cfu/ml, about 5×10³ cfu/ml, 1×10⁴ cfu/ml, about 2.5×10⁴ cfu/ml, about 5×10⁴ cfu/ml, 1×10⁵ cfu/ml, about 2.5×10⁵ cfu/ml, about 5×10⁵ cfu/ml, 1×10⁶ cfu/ml, about 2.5×10⁶ cfu/ml, about 5×10⁶ cfu/ml, 1×10⁷ cfu/ml, about 2.5×10⁷ cfu/ml, about 5×10⁷ cfu/ml, 1×10⁸ cfu/ml, about 2.5×10⁸ cfu/ml, about 5×10⁸ cfu/ml, 1×10⁹ cfu/ml, about 2.5×10⁹ cfu/ml, or about 5×10⁹ cfu/ml, about 1×10¹⁰ cfu/ml, about 1.5×10¹⁰ cfu/ml, about 2.5×10¹⁰ cfu/ml, about 5×10¹⁰ cfu/ml or about 1×10¹¹ cfu/ml.

Also contemplated by the present disclosure is the use of variants of the Lactobacillus species described herein. As used herein, the term “variant” refers to both naturally occurring and specifically developed variants or mutants of the species disclosed and exemplified herein. Variants may or may not have the same identifying biological characteristics of the specific species exemplified herein, provided they share similar advantageous properties in terms of treating constipation, treating at least one symptom of constipation or improving at least one symptom of constipation. Illustrative examples of suitable methods for preparing variants exemplified herein include, but are not limited to, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microbial strains phylogenetically closely related to species disclosed herein and strains possessing substantial sequence identity with the species disclosed herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” strain as contemplated herein may share about 85%, 86%, 87%, 88%, 89%, 90%, 91%, 92%, 93%, 94%, 95%, 96%, 97%, 98% or 99% sequence identity with a strain disclosed herein.

The Lactobacillus species described herein, and combinations thereof, or culture supernatants or cell free filtrates derived from culture media are typically administered in accordance with the present disclosure in the form of a composition. In embodiments in which combinations of species, or culture supernatants or cell free filtrates derived from culturing multiple species, those skilled in the art will appreciate that each of the species, supernatants or filtrates to be administered need not be contained in the same composition. Where administration is separate, administration may be sequential or simultaneous.

Compositions for use in accordance with the present disclosure may be prepared by admixing the relevant components and formulating the resulting mixture into a dosage form that is suitable for administration to a subject. Accordingly, the compositions may comprise pharmaceutically acceptable carriers, diluents, excipients and/or adjuvants. The carriers, diluents, excipients and adjuvants must be “acceptable” in terms of being compatible with other components of the composition, and not deleterious to the subject who is to receive the composition.

Compositions for administration in accordance with the present disclosure may be administered in any suitable form, typically by oral administration. The composition may be in unit dosage form. The term “unit dose” refers to a physically discrete unit of a dosage form suitable for use in humans, each unit containing a predetermined quantity of the composition to provide an effective amount. The compositions may be administered in any suitable form, typically in solid or liquid form. For example, the compositions may be formulated using methods and techniques well known to those skilled in the art, into tablets, troches, capsules, caplets, elixirs, suspensions, syrups, wafers, granules, powders, gels, pastes, solutions, creams, sprays, suspensions, soluble sachets, lozenges, effervescent tablets, chewable tablets, multi-layer tablets, and the like.

By way of example the composition may be provided to the user in a powder form, suitable for mixing by the user into any type of drink or food product (for example water, fruit juice or yoghurt) or for consumption as a powder in the absence of a drink or additional food product. The composition may be conveniently incorporated in a variety of food and/or beverage products, nutraceutical products, supplements, food additives, and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

In particular embodiments, compositions to be administered in accordance with the present disclosure may be used as nutritional supplements.

As will be appreciated by those skilled in the art, the choice of pharmaceutically acceptable carrier or diluent may be readily determined by a person skilled in the art. A person skilled in the art will readily be able to determine appropriate formulations useful in the methods of the disclosure using conventional approaches.

Solid forms for oral administration may contain binders acceptable in pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, stevia, xylitol, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof. Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or actyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. emulsions for oral administration may further comprise one or more emulsifying agents. Suitable emulsifying agents include dispersing agents as exemplified above or natural gums such as guar gum, gum acacia or gum tragacanth.

In exemplary embodiments, each dose of a liquid composition may comprise about 1 ml to about 25 ml liquid formulation of Lactobacillus paracasei, Lactobacillus zeae and Lactobacillus diolivorans at a final concentration of between about 10⁵ and 10¹¹ cfu/ml. The volume of the liquid formulation may be, for example, about 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, or 25 ml.

Compositions for use in accordance with the present disclosure may comprise one or more prebiotic components. Suitable prebiotics include, for example, polydextrose, inulin, fructooligosaccharides (FOS), xylooligosaccharides (XOS), galactooligosaccharides (GOS), mannan oligosaccharides, protein-based green lipped mussel extract, and various prebiotic-containing foods such as raw onion, raw leek, raw chickory root and raw artichoke. In certain embodiments the prebiotic is a fructooligosaccharide.

In certain embodiments, the methods of the present disclosure may be employed as part of a broader regime to treat constipation and to improve a subject's bowel movements and overall digestive and gastrointestinal health. As such, the present methods may be used in conjunction with other methods known to treat constipation or symptoms of constipation. Additionally, compositions for use in accordance with the present disclosure may incorporate additional ingredients known to treat constipation, to improve symptoms of constipation, to improve a subject's bowel movements and/or to improve a subject's overall digestive and gastrointestinal health.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1

A phase I study was conducted in adult human subjects with chronic functional constipation using a live biotherapeutic product comprising Lactobacillus paracasei, Lactobacillus zeae and Lactobacillus diolivorans. The biotherapeutic product was administered orally twice daily for a period of 8 weeks in a 10 mL dose comprising 1.5×10¹⁰ CFU/dose of L. paracasei, L. zeae and L. diolivorans (0.5×10⁹ CFU/dose of each of L. paracasei SVT 04P1, L. zeae SVT 08Z1 and L. diolivorans SVT 01D1) suspended in 0.9% saline. Other excipients in the composition include synthetic raspberry flavour (0.3%), stevia (0.01%) and purified water.

Participants were recruited from male and female adults ≥18 years of age. Inclusion criteria included confirmation of diagnosis of idiopathic constipation or constipation-predominant irritable bowel syndrome according to the Rome III Diagnostic Criteria (e.g. Longstreth et al., 2006, Gastroenterology 130:1480-1491). Exclusion criteria included: diarrhoea-predominant or alternating irritable bowel syndrome; immunocompromised or immunodeficiency; history of anti-TNF treatment or other immunosuppressant medications; current ≥15 mg/daily or oral prednisolone (or equivalent) use and/or a history of intermittent corticosteroid usage ≥40 mg/daily or oral prednisolone (or equivalent) of >3 days in the last 3 months; and use of oral antibiotics within 2 weeks of baseline visit (Day 0) and for the duration of the study.

Participants completed two validated, self-administered Patient Assessment of Constipation (PAC) questionnaires at baseline (day 0), at week 4 (mid-treatment), at week 8 (end of treatment and again 4 weeks after the end of treatment (at week 12).

The Patient Assessment of Constipation-Symptoms (PAC-SYM) questionnaire (Frank et al., 1999, Scand J Gastroenterol 34:870-877) is divided into three symptom groups, abdominal, rectal and stool, each scored on a 5-point scale of severity. A minimal important difference (MID) of −0.6 (i.e. a reduction in total PAC-SYM score of at least 0.6) or −0.75 was used as a threshold to define a positive response to treatment. A MID threshold of −0.6 is consistent with that recommended as a reduction in PAC-SYM score to define a clinical response, whereas a MID threshold of −0.75 may be used in clinical trials to reduce the placebo response rate (Yiannakou et al., 2017, Ailment Pharmacol Ther 46:1103-1111). Results are shown in Table 1.

TABLE 1
PAC-SYM scores and MID thresholds

| | Week 4 (mid- | | Week 8 (end of | | | |
|---|---|---|---|---|---|---|
| | treatment) | | treatment) | | Week 12 | |
| | MID | MID | MID | MID | MID | MID |
| | 0.6¹ | 0.75² | 0.6¹ | 0.75² | 0.6¹ | 0.75² |
| Avg change in | 0.52 | | 0.69 | | 0.69 | |
| score | | | | | | |
| Number of | 24 | 23 | 21 | 20 | 16 | 15 |
| participants | | | | | | |
| % participants | 42% | 39% | 38% | 35% | 38% | 33% |
| meeting MID | | | | | | |
| threshold | | | | | | |

¹Threshold score reduction of 0.6
²Threshold score reduction of 0.75

The Patient Assessment of Constipation-Quality of Life (PAC-QOL) questionnaire (Dubois et al., 2010, Neurogastroenterol Motil 22 e54-63) provides a measure of the impact and burden of constipation on a constipated patient's everyday life. Questions are categorised to 4 items on physical discomfort, 8 items on psychosocial discomfort, 5 items on treatment satisfaction, and 11 items on worries and discomfort, each scored on a 5-point scale of severity. A MID of −0.5 (i.e. a reduction in total PAC-QOL score of at least 0.5) was used as a threshold to define a positive response to treatment (Marquis et al., 2005, Scand J Gastroenterol 40:540-551). Results are shown in Table 2.

TABLE 2
PAC-QOL scores and MID thresholds

| | Week 4 (mid- | Week 8 (end of | |
|---|---|---|---|
| | treatment) | treatment) | Week 12 |
| | MID 0.5¹ | MID 0.5¹ | MID 0.5¹ |
| Avg change in | 0.59 | 0.78 | 0.79 |
| score | | | |
| Number of | 24 | 19 | 14 |
| participants | | | |
| % participants | 54% | 68% | 57% |
| meeting MID | | | |
| threshold | | | |

¹Threshold score reduction of 0.5

The beneficial effect of treatment on participants' quality of life is evident upon analysis of subcategory scores of PAC-QOL as shown in Tables 3 to 6 below.

TABLE 3
PAC-QOL scores (physical discomfort) and MID thresholds

| | Week 4 (mid- | Week 8 (end of | |
|---|---|---|---|
| | treatment) | treatment) | Week 12 |
| | MID 0.5 | MID 0.5 | MID 0.5 |
| Avg change in | 0.60 | 0.74 | 0.82 |
| score | | | |
| Number of | 24 | 19 | 14 |
| participants | | | |
| % participants | 58% | 42% | 64% |
| meeting MID | | | |
| threshold | | | |

TABLE 4
PAC-QOL scores (psychosocial discomfort) and MID thresholds

| | Week 4 (mid- | Week 8 (end of | |
|---|---|---|---|
| | treatment) | treatment) | Week 12 |
| | MID 0.5 | MID 0.5 | MID 0.5 |
| Avg change in | 0.61 | 0.88 | 0.84 |
| score | | | |
| Number of | 24 | 19 | 14 |
| participants | | | |
| % participants | 50% | 68% | 57% |
| meeting MID | | | |
| threshold | | | |

TABLE 5
PAC-QOL scores (worries/concerns) and MID thresholds

| | Week 4 (mid- | Week 8 (end of | |
|---|---|---|---|
| | treatment) | treatment) | Week 12 |
| | MID 0.5 | MID 0.5 | MID 0.5 |
| Avg change in | 0.51 | 0.71 | 0.77 |
| score | | | |
| Number of | 24 | 19 | 14 |
| participants | | | |
| % participants | 38% | 58% | 50% |
| meeting MID | | | |
| threshold | | | |

TABLE 6
PAC-QOL scores (satisfaction) and MID thresholds

| | Week 4 (mid- | Week 8 (end of | |
|---|---|---|---|
| | treatment) | treatment) | Week 12 |
| | MID 0.5 | MID 0.5 | MID 0.5 |
| Avg change in | 0.77 | 0.74 | 0.72 |
| score | | | |
| Number of | 24 | 19 | 14 |
| participants | | | |
| % participants | 63% | 78% | 64% |
| meeting MID | | | |
| threshold | | | |

### Example 2

A further phase I study was conducted in adult human subjects diagnosed, according to the Rome III Diagnostic Criteria, with idiopathic constipation (IC), constipation-predominant irritable bowel syndrome (IBS-C) or mixed diagnosis of both constipation and diarrhoea (IBS-M), using the live biotherapeutic product described in Example 1. Participants were recruited from male and female adults ≥18 years of age. Participants were excluded if they had diarrhoea-predominant irritable bowel syndrome, were immunocompromised, had used oral antibiotics or corticosteroids 2 to 12 weeks prior to the baseline visit, respectively, or were pregnant or breastfeeding. Of 38 participants enrolled, 32 were female, 17 had a diagnosis of IBS-C, 10 of IC and 11 of IBS-M. The average age of participants was 49 years, and the mean BMI 26.5 (±5.8).

Participants were administered the biotherapeutic product orally twice daily for a period of 8 weeks in a 10 mL dose comprising 1.5×10¹⁰ CFU/dose of L. paracasei, L. zeae and L. diolivorans (0.5×10⁹ CFU/dose of each of L. paracasei SVT 04P1, L. zeae SVT 08Z1 and L. diolivorans SVT 01D1) suspended in 0.9% saline, as described in Example 1. Participants completed PAC-SYM and PAC-QOL questionnaires at baseline (day 0), at week 4 (mid-treatment), at week 8 (end of treatment and again 4 weeks after the end of treatment (at week 12). Of the 38 participants enrolled into the study, 32 completed to week 4, 29 completed to week 8, and 22 completed to week 12. The PAC-SYM and PAC-QOL questionnaires are described in Example 1.

Administration of the biotherapeutic product was determined to be extremely safe and tolerable. Expected events occurred due to the nature of constipation, which included pre-existing symptoms of flatulence, abdominal bloating and cramping and nausea. Blood pathology, biochemistry and liver function tests demonstrated no adverse outcomes.

A total of 15 participants provided both a baseline and week 12 completed Rome III criteria questionnaire. There was a statistically significant improvement (p=0.0027) in the Rome III criteria scores with a mean decrease in score of 9.80 (SE=2.696). Analysis of the Rome III criteria at week 12 indicated two participants had sufficient resolution of symptoms to no longer meet the criteria for any IBS subtype or IC.

Mean PAC-SYM scores at each time point are shown in Table 7. The difference of least mean squares for PAC-SYM are shown in Table 8.

PAC-SYM scores for abdominal symptoms indicate that the baseline, week 4, 8 and 12 means (SE) were respectively 2.00 (0.16), 1.52 (0.17), 1.43 (0.17), 1.40 (0.20). The pairwise treatment comparisons show that week 4, 8 and 12 means are significantly less than the baseline mean (p≤0.01). PAC-SYM scores for rectal symptoms indicate that the baseline, week 4, 8 and 12 means (SE) were respectively 1.43 (0.15), 1.04 (0.16), 0.76 (0.17), 0.91 (0.19). The pairwise treatment comparisons show that week 4, 8 and 12 means are significantly less than the baseline mean (p≤0.02). PAC-SYM for stool symptoms indicate that the baseline, week 4, 8 and 12 means (SE) were respectively 2.31 (0.15), 1.82 (0.16), 1.73 (0.17), 1.82 (0.19). The pairwise treatment comparisons show that week 4, 8 and 12 means are significantly less than the baseline mean (p≤0.02). PAC-SYM total average scores indicate that the baseline, week 4, 8 and 12 means (SE) were respectively 2.04 (0.12), 1.57 (0.13), 1.46 (0.14), 1.51 (0.15). The pairwise treatment comparisons show that week 4, 8 and 12 means are significantly less than the baseline mean (p≤0.002).

A minimal important difference (MID) of −0.5 (i.e. a reduction in total PAC-SYM score of at least 0.5) was used as a threshold to define a positive response to treatment. A 1.0 point change corresponds to a moderate improved difference. There was a statistically significant improvement in overall PAC-SYM scores compared to baseline at week 4 and week 8 (p<0.001) that continued through to week 12 (p=0.002) (see Table 8). It was observed that 14 out of 38 participants as intention to treat population, whose decrease from baseline compared to week 8 was greater than or equal to the MID of 0.5 (p<0.001). From the per protocol population, 14 out of 29 were observed (p<0.001).

TABLE 7
Least Square means for PAC-SYM scores

| Parameter | Week | Mean | SE |
|---|---|---|---|
| Abdominal Symptoms | 0 | 2.00 | 0.16 |
| | 4 | 1.52 | 0.17 |
| | 8 | 1.43 | 0.17 |
| | 12 | 1.40 | 0.20 |
| Rectal Symptoms | 0 | 1.43 | 0.15 |
| | 4 | 1.04 | 0.16 |
| | 8 | 0.76 | 0.17 |
| | 12 | 0.91 | 0.19 |
| Stool Symptoms | 0 | 2.31 | 0.15 |
| | 4 | 1.82 | 0.16 |
| | 8 | 1.73 | 0.17 |
| | 12 | 1.82 | 0.19 |
| Total Average | 0 | 2.04 | 0.12 |
| | 4 | 1.57 | 0.13 |
| | 8 | 1.46 | 0.14 |
| | 12 | 1.51 | 0.15 |

TABLE 8
Difference of Least Square Means for
PAC-SYM at MID threshold of 0.5

| | Difference | | | |
|---|---|---|---|---|
| Parameter | between weeks | Mean | SE | P-value |
| Abdominal Symptoms | 0 vs 4 | 0.48 | 0.15 | 0.002 |
| | 0 vs 8 | 0.57 | 0.19 | 0.004 |
| | 0 vs 12 | 0.60 | 0.23 | 0.010 |
| | 4 vs 8 | 0.09 | 0.16 | 0.554 |
| | 4 vs 12 | 0.12 | 0.21 | 0.578 |
| | 8 vs 12 | 0.03 | 0.18 | 0.885 |
| Rectal Symptoms | 0 vs 4 | 0.39 | 0.16 | 0.014 |
| | 0 vs 8 | 0.67 | 0.20 | <0.001 |
| | 0 vs 12 | 0.53 | 0.23 | 0.023 |
| | 4 vs 8 | 0.28 | 0.16 | 0.091 |
| | 4 vs 12 | 0.14 | 0.22 | 0.530 |
| | 8 vs 12 | −0.14 | 0.19 | 0.441 |
| Stool Symptoms | 0 vs 4 | 0.48 | 0.13 | <0.001 |
| | 0 vs 8 | 0.57 | 0.17 | 0.001 |
| | 0 vs 12 | 0.49 | 0.21 | 0.022 |
| | 4 vs 8 | 0.09 | 0.14 | 0.495 |
| | 4 vs 12 | 0.01 | 0.19 | 0.967 |
| | 8 vs 12 | −0.09 | 0.16 | 0.583 |
| Total Average | 0 vs 4 | 0.47 | 0.10 | <0.001 |
| | 0 vs 8 | 0.59 | 0.14 | <0.001 |
| | 0 vs 12 | 0.54 | 0.17 | 0.002 |
| | 4 vs 8 | 0.12 | 0.11 | 0.288 |
| | 4 vs 12 | 0.06 | 0.15 | 0.675 |
| | 8 vs 12 | −0.05 | 0.12 | 0.672 |

Mean PAC-QOL scores at each time point are shown in Table 9. The difference of least mean squares for PAC-QOL are shown in Table 10. The beneficial effect of treatment on participants' quality of life is evident upon analysis of subcategory scores of PAC-QOL as shown in Tables 9 and 10.

PAC-QOL scores for physical discomfort indicate that the baseline, week 4, 8 and 12 means (SE) were respectively 2.24 (0.16), 1.63 (0.17), 1.55 (0.18), 1.45 (0.20). Pairwise treatment comparisons show that week 4, 8 and 12 means are significantly less than the baseline mean (p≤0.001). PAC-QOL scores for psychological discomfort indicate that the baseline, week 4, 8 and 12 means (SE) were respectively 1.51 (0.15), 0.92 (0.16), 0.82 (0.17), 0.95 (0.19). The pairwise treatment comparisons show that week 4, 8 and 12 means are significantly less than the baseline mean (p≤0.006). PAC-QOL scores for worries/concerns indicate that the baseline, week 4, 8 and 12 means (SE) were respectively 2.16 (0.17), 1.63 (0.18), 1.60 (0.19), 1.67 (0.21). The pairwise treatment comparisons show that week 4, 8 and 12 means are significantly less than baseline mean (p<0.05). PAC-QOL for satisfaction indicate that the baseline, week 4, 8 and 12 means (SE) were respectively 3.15 (0.15), 2.36 (0.16), 2.36 (0.17) and 2.52 (0.19). The pairwise treatment comparisons show that week 4, 8 and 12 means are significantly less than baseline mean (p≤0.009). PAC-QOL total average scores indicate that the baseline, week, 4, 8 and 12 means (SE) were respectively 2.16 (0.14), 1.56 (0.14), 1.51 (0.15), 1.59 (0.17). The pairwise treatment comparisons show that week 4, 8 and 12 means are significantly less than the baseline mean (p≤0.003).

A MID of −0.5 (i.e. a reduction in total PAC-QOL score of at least 0.5) was used as a threshold to define a positive response to treatment. There was a statistically significant improvement in overall PAC-QOL scores compared to baseline at week 4 and week 8 (p<0.001) that continued through to week 12 (p=0.003) (see Table 10). It was observed that 18 out of 38 participants as intention to treat population whose decrease from baseline compared to week 8 was greater than or equal to the MID of 0.5 (p<0.001). From the per protocol population, 18 out of 29 were observed (P<0.001).

TABLE 9
Least Square Means for PAC-QOL

| Parameter | Week | Mean | SE |
|---|---|---|---|
| Physical Discomfort | 0 | 2.24 | 0.16 |
| | 4 | 1.63 | 0.17 |
| | 8 | 1.55 | 0.18 |
| | 12 | 1.45 | 0.20 |
| Psychological Discomfort | 0 | 1.51 | 0.15 |
| | 4 | 0.92 | 0.16 |
| | 8 | 0.82 | 0.17 |
| | 12 | 0.95 | 0.19 |
| Worries/Concerns | 0 | 2.16 | 0.17 |
| | 4 | 1.63 | 0.18 |
| | 8 | 1.60 | 0.19 |
| | 12 | 1.67 | 0.21 |
| Satisfaction | 0 | 3.15 | 0.15 |
| | 4 | 2.36 | 0.16 |
| | 8 | 2.36 | 0.17 |
| | 12 | 2.52 | 0.19 |
| Total Average | 0 | 2.16 | 0.14 |
| | 4 | 1.56 | 0.14 |
| | 8 | 1.51 | 0.15 |
| | 12 | 1.59 | 0.17 |

TABLE 10
Difference of Least Squares Means for PAC-QOL

| | Difference | | | |
|---|---|---|---|---|
| Parameter | between weeks | Mean | SE | P-value |
| Physical Discomfort | 0 vs 4 | 0.61 | 0.15 | <0.001 |
| | 0 vs 8 | 0.70 | 0.20 | <0.001 |
| | 0 vs 12 | 0.79 | 0.23 | 0.001 |
| | 4 vs 8 | 0.09 | 0.16 | 0.596 |
| | 4 vs 12 | 0.18 | 0.22 | 0.411 |
| | 8 vs 12 | 0.10 | 0.18 | 0.602 |
| Psychological Discomfort | 0 vs 4 | 0.59 | 0.12 | <0.001 |
| | 0 vs 8 | 0.69 | 0.16 | <0.001 |
| | 0 vs 12 | 0.56 | 0.20 | 0.006 |
| | 4 vs 8 | 0.10 | 0.13 | 0.422 |
| | 4 vs 12 | −0.03 | 0.18 | 0.878 |
| | 8 vs 12 | −0.13 | 0.15 | 0.370 |
| Worries/Concerns | 0 vs 4 | 0.53 | 0.14 | <0.001 |
| | 0 vs 8 | 0.56 | 0.19 | 0.005 |
| | 0 vs 12 | 0.49 | 0.23 | 0.038 |
| | 4 vs 8 | 0.03 | 0.15 | 0.842 |
| | 4 vs 12 | −0.04 | 0.21 | 0.861 |
| | 8 vs 12 | −0.07 | 0.17 | 0.696 |
| Satisfaction | 0 vs 4 | 0.79 | 0.17 | <0.001 |
| | 0 vs 8 | 0.79 | 0.21 | <0.001 |
| | 0 vs 12 | 0.63 | 0.24 | 0.009 |
| | 4 vs 8 | 0.00 | 0.18 | 0.990 |
| | 4 vs 12 | −0.16 | 0.23 | 0.485 |
| | 8 vs 12 | −0.16 | 0.20 | 0.427 |
| Total Average | 0 vs 4 | 0.60 | 0.12 | <0.001 |
| | 0 vs 8 | 0.65 | 0.15 | <0.001 |
| | 0 vs 12 | 0.57 | 0.19 | 0.003 |
| | 4 vs 8 | 0.05 | 0.12 | 0.663 |
| | 4 vs 12 | −0.03 | 0.17 | 0.856 |
| | 8 vs 12 | −0.08 | 0.14 | 0.544 |

### Deposit Details

Details of the biological material deposited pursuant to the Budapest Treaty are provided hereinbefore in the specification.

The following Lactobacilli have been described previously, for example in WO 2020/073088, the disclosure of which is incorporated herein. In summary:
Lactobacillus paracasei SVT 04P1 was deposited pursuant to the Budapest Treaty with the Belgian Coordinated Collections of Micro-organisms (BCCM), Federal Public Planning Service Science Policy, 8 rue de la Science B-1000, Brussels, Belgium, on 27 Feb. 2019 under Accession Number LMG P-31290.
Lactobacillus zeae SVT 08Z1 was deposited pursuant to the Budapest Treaty with the Belgian Coordinated Collections of Microorganisms (BCCM), Federal Public Planning Service Science Policy, 8 rue de la Science B-1000, Brussels, Belgium, on 27 Feb. 2019 under Accession Number LMG P-31295.
Lactobacillus diolivorans SVT 01D1 was deposited pursuant to the Budapest Treaty with the Belgian Coordinated Collections of Microorganisms (BCCM), Federal Public Planning Service Science Policy, 8 rue de la Science B-1000, Brussels, Belgium, on 27 Feb. 2019 under Accession Number LMG P-31287.
Lactobacillus parafarraginis SVT 05P2 was deposited pursuant to the Budapest Treaty with the Belgian Coordinated Collections of Microorganisms (BCCM), Federal Public Planning Service Science Policy, 8 rue de la Science B-1000, Brussels, Belgium, on 27 Feb. 2019 under Accession Number LMG P-31292.
Lactobacillus buchneri SVT 06B1 was deposited pursuant to the Budapest Treaty with the Belgian Coordinated Collections of Microorganisms (BCCM), Federal Public Planning Service Science Policy, 8 rue de la Science B-1000, Brussels, Belgium, on 27 Feb. 2019 under Accession Number LMG P-31293.
Lactobacillus rapi SVT 07R1 was deposited pursuant to the Budapest Treaty with the Belgian Coordinated Collections of Microorganisms (BCCM), Federal Public Planning Service Science Policy, 8 rue de la Science B-1000, Brussels, Belgium, on 27 Feb. 2019 under Accession Number LMG P-31294.

Lactobacillus plantarum SVT 09P1 was deposited pursuant to the Budapest Treaty with the Belgian Coordinated Collections of Microorganisms (BCCM), Federal Public Planning Service Science Policy, 8 rue de la Science B-1000, Brussels, Belgium, on 10 Aug. 2020 under Accession Number LMG P-31923 and has been described previously in PCT/AU2021/051432, the disclosure of which is incorporated herein.

1. A method for treating constipation in a subject, comprising administering to the subject an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

2. A method according to claim 1, wherein the constipation is functional constipation.

3. A method according to claim 1, wherein the constipation is chronic constipation.

4. A method according to claim 1, wherein the subject has idiopathic constipation.

5. A method according to claim 1, wherein the subject has constipation-predominant irritable bowel syndrome.

6. A method according to claim 1, comprising administering a composition comprising Lactobacillus paracasei, Lactobacillus zeae and Lactobacillus diolivorans.

7. A method according to claim 6, wherein the composition is in the form of a liquid or solid unit dosage form, a food or a beverage.

8. A method according to claim 1, further comprising administering one or more additional agents selected from Lactobacillus buchneri, Lactobacillus rapi, Lactobacillus plantarum, and Lactobacillus parafarraginis, or culture supernatant(s) or cell free filtrate(s) derived from culture media in which said Lactobacillus species have been cultured.

9. A method for treating at least one symptom of constipation in a subject with constipation, comprising administering to the subject an effective amount of a combination of Lactobacillus paracasei, Lactobacillus zeae and Lactobacillus diolivorans or culture supernatant(s) or cell free filtrate(s) derived from culture media in which said Lactobacillus species have been cultured.

10. A method according to claim 9, wherein the at least one symptom of constipation comprises abdominal discomfort, abdominal pain, abdominal bloating, abdominal cramping, painful bowel movements, rectal burning during or after a bowel movement, incomplete bowel movements, straining to pass a bowel movement and/or inability to pass a bowel movement.

11. A method according to claim 9, wherein the constipation is functional constipation.

12. A method according to claim 9, wherein the constipation is chronic constipation.

13. A method according to claim 9, wherein subject has idiopathic constipation.

14. A method according to claim 9, wherein the subject has constipation-predominant irritable bowel syndrome.

15. A method according to claim 9, comprising administering a composition comprising Lactobacillus paracasei, Lactobacillus zeae and Lactobacillus diolivorans.

16. A method according to claim 15, wherein the composition is in the form of a liquid or solid unit dosage form, a food or a beverage.

17. A method according to claim 9, further comprising administering one or more additional agents selected from Lactobacillus buchneri, Lactobacillus rapi, Lactobacillus plantarum, and Lactobacillus parafarraginis, or culture supernatant(s) or cell free filtrate(s) derived from culture media in which said Lactobacillus species have been cultured.
